(19) United States
(12) Patent Application Publication
(10) Pub. No.: US 2018/0344216 A1
(43) Pub. Date: Dec. 6, 2018

(54) Indication Device

(71) Applicant: Pinpoint Biomechanics Holding IVS, Ebeltoft (DK)

(72) Inventor: Finn Christian Simonsen, Ebeltoft (DK)

(21) Appl. No.: 15/780,267

(22) PCT Filed: Nov. 29, 2016
(86) PCT No.: PCT/DK2016/050402

§ 371 Date: May 31, 2018

(30) Foreign Application Priority Data
Dec. 1, 2015 (DK) PA 2015 00772
Dec. 17, 2015 (DK) PA 2015 00816
Feb. 25, 2016 (DK) PA 2016 70105
Oct. 4, 2016 (DK) PA 2016 70784

(51) Int. Cl. A61B 5/11; A61B 5/107; A61B 5/00

(52) U.S. Cl. CPC A61B 5/1121; A61B 5/1077; A61B 5/6832; A61B 5/6829; A61B 5/1127; A61B 5/4595

(57) ABSTRACT

Indication device comprising an elongated indication pin connected to a base, said base suitable to be arranged on or to the surface of a body.

### FIELD OF THE INVENTION

The present invention relates to an indication device as well as a method of using an indication device. The indication device is particularly designed to be applied to the surface of a body and in particular the indication device is used for investigating any articulated joint in a body be it the body of a human being or the body of an animal.

### BACKGROUND OF THE INVENTION

A skeleton, be it a skeleton of a human being or an animal comprises a vast number of joints which nature has designed to function perfectly such that the body will be able to move effortless and painlessly according to the design of the body. However, in some situations misalignment of body parts occurs for example due to deformations, broken limbs, monotonous wear etc. etc.

In order to compensate for this, the body is usually examined by an expert be it a physiotherapist, a doctor or veterinarian who due to his or her experience and judgment decides on measures to be taken in order to compensate and correct the misalignment or deformation such that proper treatment, therapy or use of devices may be implemented in order to compensate or alleviate the misalignment. Basically the basis for the entire procedure is the subjective judgement of the expert treating that particular body.

Typically an expert will judge and evaluate the situation from the expert's experience, and as such a physiotherapist may view and evaluate a situation differently from a medical expert due to the difference in background and knowledge. This may cause vastly different treatments and success rates.

Particularly for the examination of the human foot there is a need for particular attention, as the foot/ankle comprises many articulated joints.

In the physiological analysis and treatment of various conditions related to the position of various parts of the foot, it is relevant and necessary to perform a qualitative or quantitative measurement of the actual scope of the condition.

In relation to more common daily routines as selecting the right footwear for a specific type of foot alignment during for example running it is likewise relevant to make a qualitative or quantitative analysis of the actual condition in order to ensure the right choice or recommendation regarding footwear.

Previously it has been a normal procedure to provide an indication by making a marking, e.g. a line, on a person's foot and lower leg and following this during movement, either entirely visually by eye or by video recording, eventually assisted by kinematic or kinetic systems. This has worked to some extent, but a significant uncertainty is connected with this measurement and hence a need for improvement is present.

The objective of the present invention is to provide an improved indicator that will enable an easier and more reliable analysis of the foot motion.

### OBJECT OF THE INVENTION

Consequently, it is an object of the present invention to provide a device which facilitates a more objective judgement of the status of the articulated joint or joint complex or involved body segments.

### DESCRIPTION OF THE INVENTION

The invention addresses this by providing an indication device comprising an elongated indication pin connected to a base, said base suitable to be arranged on or to the surface of a body.

In order to ensure a proper adhesion to the surface of a body any body hair/fur should be removed in the desired area and any moisture or other substances should be cleaned as well in order to ensure proper adhesion to the surface of the body.

During the application stage of the indication device to the body the practitioner's experience is important in that the indication pin shall be aligned correctly with respect to that particular joint or joint complex or particular complex of body segments which is being examined.

In a further advantageous embodiment of the invention the indication device further comprises a scale extending from the base and arranged on the elongate element and/or behind the indication pin, where markings are provided on the scale facing the indication pin.

The scale will be able to give an objective reading of the relative angle between the two body parts on either side of the articulated joint. This reading is of course dependent on the practitioner's proper application of the indication device. However, it is contemplated that the scale may be the same for indication devices, for example used in order to investigate the ankle joint such that the reading on the scale if so desired may be the same for all ankle joint investigations and thereby it becomes possible to compare results from one investigation with results from another investigation.

In this manner it is possible to generate a number or numbers on the scale or a zone on the scale which may be denoted as normal and likewise, if the indication pin is in certain positions relative to the scale, this may be denoted abnormal and requiring treatment.

In a further advantageous embodiment the scale at least in certain areas may be provided with adhesive means suitable to adhere the scale to the surface of the body.

Often the investigation of the articulated joint requires movement in order to investigate the joint's spatial position or movement and consequently if the scale was completely loose the scale may move with the indication pin and thereby not provide the correct readings whereas if the scale is adhered to the joint and in particular on the other side of the joint as to where the base of the indication device is adhered, the relative movement between the scale and the indication pin will give a proper and correct reading.

It is also contemplated to mark the scale on the body's surface, for example as a temporary tattoo, transfer colouring, or spray painted through a stencil.

In a further advantageous embodiment of the invention the indication device is adhered to the body by an adhesive provided on the base covered by a protective cover which is removed prior to application of the device.

By providing a protective cover on the base it is possible to manufacture the indication device in large numbers, store them and send/distribute them without the adhesive losing its adhesive properties.

In a still further advantageous embodiment of the invention the base is flexible and may be deformed to fit closely to the body and where the indication pin is rigid.

In this manner it is possible to adapt the base to the body part such that a firm and solid connection is provided between the base and the body part and thereby between the indication device and the body part which it is desirable to investigate. The rigid pin naturally provides less deflection such that a reading of the relative angle between the two articulated body parts may be more precise.

In a further advantageous embodiment the base is further provided with tear lines, such that upon removal of the base, the base will tear along the tear lines. By providing tear lines the base will tear when the indication device is removed from the body part. The ensures that the device is only for one time use and consequently that the indication device specifically where the indication device is provided with scales suitable to indicate normal and not normal relative angles for specific joints is not reused such that an incorrect indication device may be applied to a wrong articulation joint and thereby provide misinformation instead of providing correct information.

Also from a hygienic point of view it is important that the device is for one-time use only. As the device comes into intimate contact with the skin of a body it is important not to re-use the device on another body as bacteria or other undesirable matter may be transferred, and thereby potentially be a cause of contamination.

In a still further advantageous embodiment of the invention the base is attached to the desired body part by percutaneous or transcutaneous fixation directly into any skeleton bone.

The two technologies, i.e. percutaneous and transcutaneous fixation may be particularly useful when the joint to be examined has been exposed to severe trauma and the body which is to be treated or that particular body part has been neutralized for example by an anaesthetic.

The indicators will in many embodiments have a cross-shape, where reference points are placed at the three distal ends of the cross (the fourth end being integral with the base).

The indication device and/or the scales may in a further advantageous embodiment be provided with active or passive indicators.

Passive indicators are indicators which clearly indicate the position of the indicator and are detectable by observation detection instrumentation or observation detection systems. Particularly in situations where a user is being filmed or in other manner registered during movement the indicators will provide a clear and objective indication of the spatial position and movement of the joint, during the particular movement of the articular joint.

Active indicators could for example be GPS transponders, RFID or other suitable indicators which emits a signal. As is the case with passive indicators, the active indicators may be registered during motion, thereby assist in mapping the movement of an articulated joint.

These analysis, involving both passive and active indicators, may be carried out in 2D or 3D (using a plurality of cameras or detectors) and by means of suitable software, map the entire spatial position or movement in 2D or 3D and time. Compared to the subjective visual analysis carried out by a practioner, 2D or 3D complete animated mapping provides an increased objective level of insight into the particular joints movements.

In a further embodiment of the invention two indicator devices are coupled, e.g. by fixed bridge(s) and/or adjustable bridge(s), where the bridges and the indicator pins forms a parallelogram or rhomboid or other geometrical formation. The two base parts of the indicator devices are e.g. to be mounted on either side of the Os Talus bone which is part of the foot's bone structure. The Os Talus bone is normally identified by the practitioner by finding the distal ends of the bone—on either side of the foot. The relative position of the Os Talus relative to the foot indicates the position of the ankle. If the Os Talus is in a neutral position (can be felt evenly on both sides of the foot) the indicator should be in the neutral position, indicating that there is neither hyperpronation nor hypersupination. If the Os Talus is translated in a lateral direction (relative to the foot) the indicator will indicate this and thereby indicate pronation or supination depending on the direction of translation.

Common for all the indicator device embodiments is the fact that the indicator device embodiments can be applied and their indications read while the person being investigated is clothed and/or wearing shoes, as the indication pin will project outside the clothing or shoes. This particular aspect is very useful. Particularly with shoes, if e.g. an insole or the shoe itself are meant to compensate for for example pronation, the effect of the insole can be objectively detected with the shoe on. At present an investigation is mainly carried out bare-footed and an estimate is used to estimate the correction needed, whereas with the present invention the result of the correction by assistive devices such as e.g. insoles or shoes or taping or by therapeutic or treatment interventions such as e.g. surgery can objectively be determined simply by reading the indication pins position on the scale.

In embodiments where a scale or measurement scale is provided, the measurement scale is being provided to estimate the movement of the elongate element, where the measurement scale is adapted to measure an angular movement of the elongate element and/or a longitudinal movement of the elongate element and/or a rotational movement of the elongate element.

Such scale may show the movement of the elongate element as an absolute measurement in either distance or in angular pivoting or in rotation or as an interval of movement, e.g. indicated with colours showing a range of displacement in either distance or angular pivoting.

The measurement scale may be adapted to be fixed onto the skin, e.g. through an adhesive applied on the measurement scale. Hereby the scale will follow the movement of the body segment e.g. the leg above the fixation of the indicator at the distal end of the elongate element.

The elongate element may be manufactured from at least two materials with different degrees of flexibility and mutually connected, e.g. through a moulding process including adhesive attachment.

In a further embodiment one or more reflective or otherwise identifiable markers are attached to the indicator's elongate element and/or the scales. Hereby the use of the indicator may be further included in motion analysis e.g. in connection with video recordings, where also markers on other body parts may be included.

As an option the attachment means are of a type allowing only single use. This will increase the sanitary conditions related to use.

The invention is also directed to a method of using an indication device as discussed above where the method utilizes the special inventive features of the present invention.

In practice the method may be carried out as follows:

When attaching the indicator it is of significant importance for the measurement result that this is done at the correct location of the foot.

The following is describing a method for measuring the inclination and/or longitudenal and/or rotational movements of Os Calcaneus in the frontal plane and the position of Os Naviculare over a substrate.

Visual observation of the hind foot:

From Posterior i.e. with visual direction from behind and towards the person.

With the direction exactly levelled with height of the Achilles tendon (Tendo Calceneo/Tendo Achillis) attachment to the heel bone (Os Calcaeneus).

With visual direction directly along the extension of the foot centerline—independent of the foot rotation in the horizontal plane in the Mid-Stand-Phase—where, if standing or walking or running on a horizontal surface, the lateral malleolus is vertically below Trochanter Major.

To do prior to visual observation:

The height of Tendo Achillis attachment to Os Calceneus relative to the substrate is measured and registered.

Posterior camera or observer's eye is adjusted in this height.

Direction of the foot center line in the horizontal plane—equal to the direction of a straight line from the heel center line to the separation between the 2. and 3. toe.

Posterior camera or observer's eye is adjusted in the extension and straight direction of this line.

Os Calceneus' sagittal center line seen in posterior view.

The observer positions himself behind the patient centrally and above the extension of the foot center line in the horizontal plane.

With left and right thumbs Os Calceneus is medially and laterally palpated as distally as possible with opposite directed and equal pressures through the skin.

A point is marked as distal as possible on Os Calceneus on the sagittal center line.

With left and right thumbs Os Calceneus is medially and laterally palpated as proximally as possible with opposite directed and equal pressures through the skin.

A point is marked as proximal as possible on Os Calceneus on the sagittal mid line.

A line is marked between the proximal and the distal points—representing the Os Calceneus' sagittal mid line.

The indicator is applied on the skin on the Os Calceneus' sagittal center line.

Os Calceneus' inclination in corrected hind foot position:

The patient is standing upright with straight knees and looking forward.

The observer places himself sitting or kneeling centrally in front of the patient.

The observer grips the patient's right knee cap with his left hand and with the right hand the left knee cap.

The observer helps the patient by use of hands and verbal instruction to rotate both patient's legs outwards in the hip joints—with extended knees.

The observer helps the patient by use of hands and verbal instruction to rotate both patient's legs inwards in the hip joints—with extended knees.

The patient repeats these movements, with help from the observer until possible to do these movements on his/her own.

The observer palpates with thumb and index finger the Os Talus necks on the patients feet while the patients conducts slow outwards and inwards rotations according to the observer's verbal instructions.

The observer palpates Os Talus neck, while it shifts medially and laterally.

The observer instructs the patient to maintain the specific rotation of his/her legs until the Os Talus neck is palpated equally medially and laterally.

The observer observes and measures with an angular measurement the inclination of Os Calcaneus' sagittal center line with the patient's hind foot in this position.

The measured inclination of Os Calceneus' sagittal center line is registered as the rear foot corrected angle.

The corrected angle is expressed in Varus (−degrees), Neutral (0 degrees) or Valgus (+degrees).

Os Calceneus' inclination in uncorrected hind foot position:

The patient relaxes from the instructed rotation of his/her legs and lets the foot rest in its uncorrected position.

The observer observes and measures the inclination of Os Calceneus' sagittal center line with the patient's hind foot in this position.

The hereby measured inclination of Os Calceneus' sagittal center line is registered as the hind foot uncorrected angle.

The uncorrected angle is expressed in Varus (−degrees), Neutral (0 degrees) or Valgus (+degrees).

Os Naviculare's height over the substrate in corrected hind foot position:

The observer instructs the patient to position and maintain the specific rotation of the legs where Os Talus necks are equally prominent when palpated medially and laterally.

The observer observes and measures with a height measurer the height of Os Naviculare over the substrate with the patient's hind foot in this position.

The hereby measured height is registered as the Os Naviculare's corrected height.

The Os Naviculare's corrected height is expressed in mm.

Os Naviculare's height over the substrate in uncorrected hind foot position:

The patient relaxes from the instructed rotation of his/her legs and lets the foot rest in its uncorrected position.

The observer observes and measures with a height measurer the height of Os Naviculare over the substrate with the patient's hind foot in this position.

The hereby measured height is registered as the Os Naviculare's uncorrected height.

The Os Naviculare's uncorrected height is expressed in mm.

Indicator is applicated on the skin on the Os Naviculare's prominence with the elongate element pointing in proximal direction.

Kinematic and/or kinetic recordings and analysis may now be performed, with or without footwear.

### DESCRIPTION OF THE DRAWING

The invention will now be explained with reference to the accompanying drawing wherein

FIG. 1a-c and 2 illustrates embodiments of an indication device

FIG. 3 illustrates a further embodiment where the device is provided with a scale on the elongate element and/or behind the elongate element

FIGS. 4 and 5 illustrates various applications to the human body

FIGS. 6 and 7 illustrates applications on a horse

FIG. 8a-c illustrates pronation of an ankle

FIG. 9a-d illustrates the device applied to the human foot from various angles and in various positions

FIG. 10 illustrates a further embodiment of the invention where two devices are connected

### DETAILED DESCRIPTION OF THE INVENTION

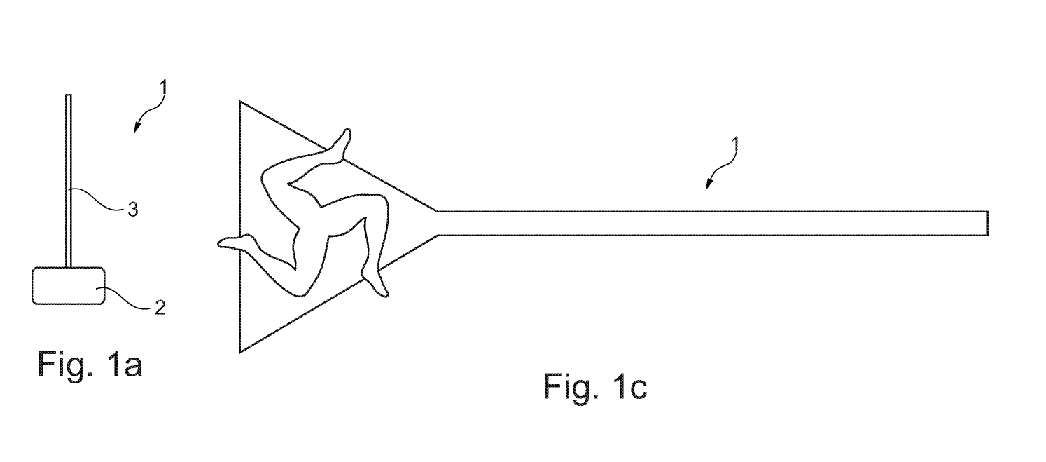

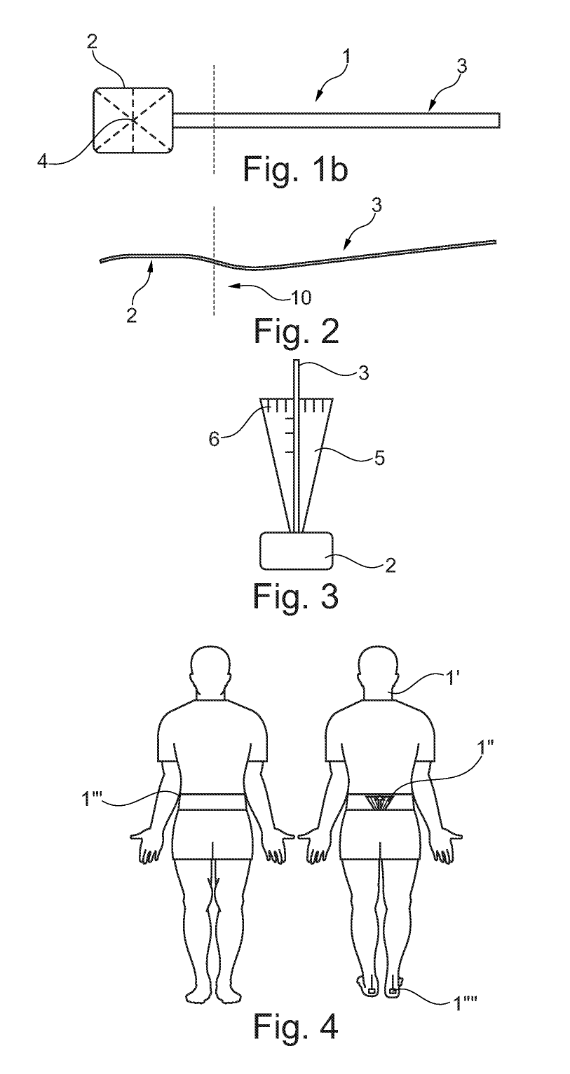

In figure 1a and b are illustrated an indication device 1 according to the present invention. The indication device 1 has a base part 2 connected to or integral with an elongated indication pin 3. In this particular embodiment depicted in figure 1b the base part 2 is provided with tear lines 4 such that when removing the indication device from a body part the base part 2 will tear and it will be necessary to use a new indication device 1 for further investigations.

Naturally the base part 2 may also be manufactured without tear lines 4 such that it is possible to retrieve the indication device without destroying the base part.

In FIG. 2 is illustrated a cross section through the indication device 1 in order to illustrate that the indication device 1 may advantageously have a curved shape. The particular shape indicated in FIG. 2 is particularly useful for investigating ankles, knees and elbows of the human body. The curvature of the base part is designed to follow the curvature of the heel (or other body part) whereas the angle of the indication pin is provided in order to make sure that the pin does not interfere with the body and as such can move freely with respect to the body part and thereby indicate the rotation relative to the fastening point of the base 2 to the body. The dashed line 10 indicates where the articulated joint should be positioned relative to the indication device 1 such that the base part 2 will be fastened on one part of the joint whereas the indication device will indicate the other part of the joint's relative angle with respect to the base part.

In FIGS. 3a and 3b is illustrated another embodiment of the present invention where the base part 2 is provided with a connected or separated scale 5. In this embodiment the scale is provided with indication marks 6 such that the relative movement of the indication pin 3 relative to the base may objectively be determined as an indication on the scale 5.

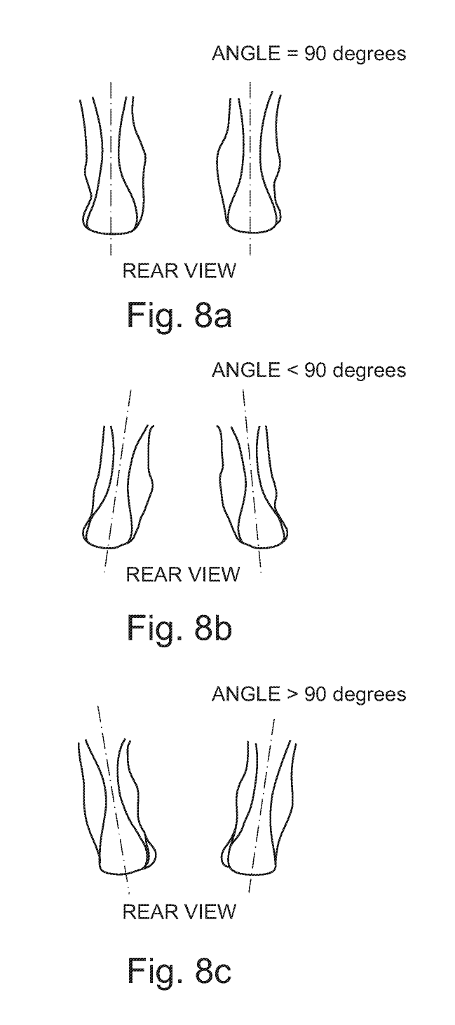

It is also contemplated that in place of the indication mark 6 the scale may be divided into coloured zones or the like such that a clear and easy reading on the scale without being tied to a specific number may be available to the user. For example if the indication devices 1 are developed in order to indicate the angle relative to the shinbone (Tibia) as will be explained further with reference to FIG. 8 the indication device may be provided in a left-foot and right-foot version where the scale includes a green area which is to considered normal and for example a yellow area where a slight deviation from normal is present and a red area where a grave deviation from normal is indicated such that it is easy for a not so experienced user to immediately detect whether or not it is necessary to investigate further and possibly start some type of treatment.

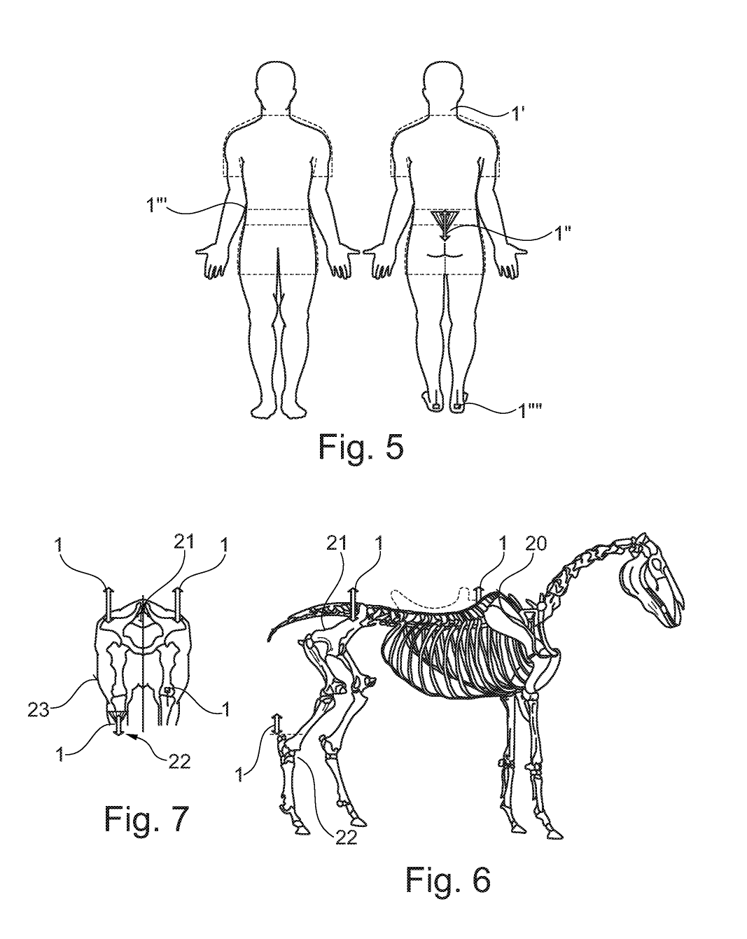

In FIGS. 4 and 5 are illustrated examples of where the indication device may be applied to a human body. The difference between FIG. 4 and FIG. 5 is the fact that the indication device according to the invention may be applied beneath the clothing of a person such that the person's personal beliefs with respect to decency, nudity and exposure may be respected without affecting the examination with an indication device according to the present invention, where the indication pin projects outside the clothing.

As the indication device may be used on any articulated joint in the body only a few examples will be mentioned. For example as illustrated in FIG. 4 and indication device 1′ may be applied on the upper part of the spine in order to indicate whether or not the neck is straight with respect to the spine.

Likewise, an indication device 1″ may be applied to the pelvis in order to estimate if the spine is correctly positioned/aligned with respect to the pelvis. Furthermore, indication devices 1′″ may be applied to the hips in order to see or detect the angle between the hip (pelvis) and the curvature of the spine.

The same indication devices are indicated in FIG. 5, however, in a situation where the person being examined is nude.

The indication device does come in various embodiments and for example one embodiment of the indication device 1′ is used without a scale for detecting the articulated joint in the neck portion whereas an indication device 1″ including a scale is used in order to indicate the relative position of the pelvis with respect to the spine.

Also in both FIGS. 4 and 5 an indication device 1″″ is provided on the heel of the patient in order to detect the situation which is illustrated with respect to FIG. 8 where the normal situation of the heel/shinbone is illustrated in FIG. 8a where over pronation is illustrated in FIG. 8b and under pronation is illustrated in FIG. 8C.

For ankles and also for certain other joints in the body indicators if they include a scale should have right and left side version being applied to respectively right and left foot, arm or shoulders such that the scale will give the correct indication.

As already mentioned above the indication device may also be used on animals and for purely illustrative purpose a skeleton of a horse is illustrated in FIGS. 6 and 7. Naturally the indication device may be used with many other animals in order to illustrate various positions relating to the articulated joints.

In this embodiment indication device 1 has been applied to the spine 20, the pelvis 21 and an ankle joint 22. As is clear from the skeleton 25 of the horse there are many articulated joints where the indication device according to the present invention may be applied in order to indicate relative positions of the bones or body segments on either side of the joint structure.

Consequently, in FIG. 7 the pelvis 21 is provided with indication devices in either side of the pelvis in order to illustrate the relative position of the pelvis for example with respect to the spine. Also in the knee joint 23 is applied an indication device 1 and the before-mentioned indication device 1 with respect to the ankle joint 22 is also illustrated.

With reference to the use on animals, as illustrated in FIGS. 7 and 8 an example is a horse where the indication device according to the invention may be very useful. For horses the horse combination or skeleton alignment and the detection of abnormalities may imply several posture and functional problems for example variation in leg length, velvet bone displacements, sacro-lliac dislocations, columnar dislocations and knee and angle joint misalignments. By applying the indicating device according to the present invention both the kinematic or the kinetic analyses may be performed such that at full access to analyze facial position and motion of the specific horse body part or body element relative to a normal condition may be performed. This also even when the particular articulated joint is covered for example by the leg of a rider or the saddle in that the indication device and in particular the indication pin may project from the saddle or from the leg of the rider etc.

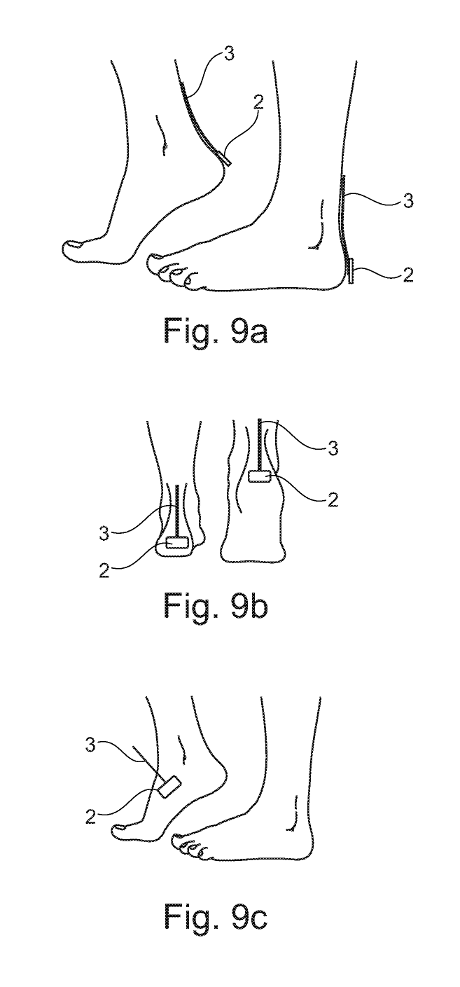

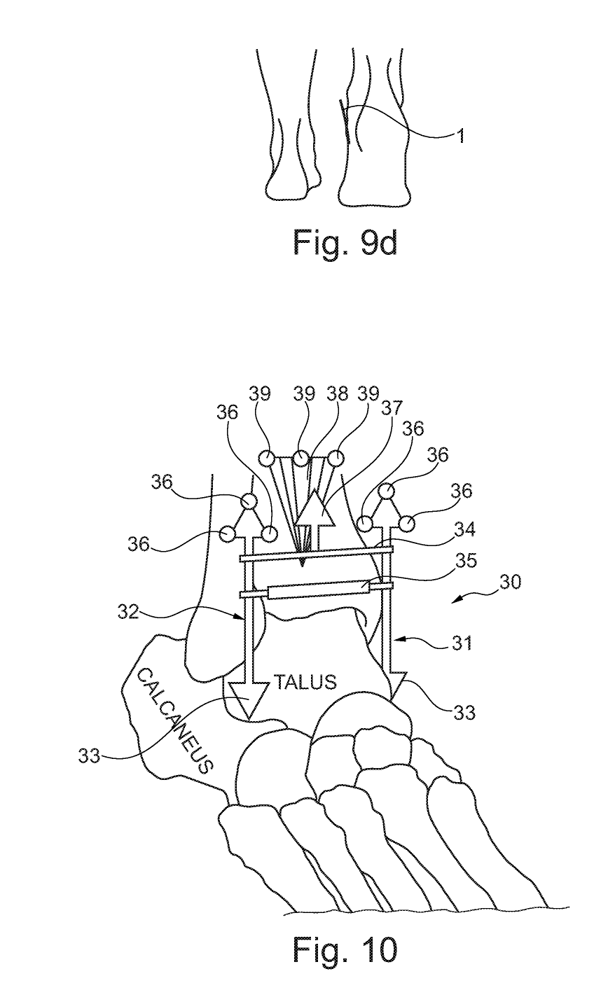

In FIGS. 9a-d are illustrated the human foot with a device according to the invention applied. As is clear from the illustrations the indicator will move, and since the base 2 of the device 1 is adhered to the outside of the foot, the movement of the indicator pin 3 is clearly identifiable as an indication of the joints' (in this illustrated example the ankle) movement relative to the base.

In FIG. 10 is illustrated a further embodiment. The indication device 30, in this embodiment comprises two indication devices 31, 32. Each indication device 31, 32 is fastened to the Os Talus (ankle bone) by a base 33. The indication devices 31, 32, are connected by two or more bridge members 34, 35. Each of the bridge members 34 and 35 may be fixed in length, or may be adjustable. In this manner the device 30 can be adjusted to fit any body structure or combination of structures, where pinch-grip contact is desired, e.g. a wide range of Os Talus' sizes.

The bridge 34 is provided with an arrow 37 which gives an indication on a scale 38 fastened on the ankle.

Furthermore, the indicator devices 31,23 are provided with active or passive indicators 36. The scale 38 is also provided with active or passive indicators 39. In this manner it is possible to detect relative movement of all the sensors and thereby of the Os Talus.

One example of using the indication device and in particular the indication device discussed above with reference to FIGS. 3a and 3b used in order to determine the ankle's position as described with reference to FIG. 8 is for the determination of for example insoles for footwear. It is contemplated within the present invention that manufactures of footwear more or less in a standard fashion manufacture insoles which addresses e.g. the pronation of a user such that for example once a user has had an examination performed using an indication device as illustrated with reference to FIGS. 3a and 3b a number of the scale 6 will be indicated and correspondingly the shoe manufacturer will provide insoles corresponding to normal standard factors such that a customer will be able to pick out a certain shoe type having a certain standard size, for example size 8, with a pronation plus or minus 1, 2 or 3 or whatever will be-come the standard.

In this manner the indication device of the present invention will have application in the consumer market and not only be adaptable for specialists investigating articulated joints.

1.-13. (canceled)

14. Indication device comprising an elongated indication pin connected to a base, said base suitable to be arranged on a body, where said indication pin can move relative to the body.

15. Indication device comprising an elongated indication pin connected to a base, said base suitable to be arranged onto the surface of a body.

16. Indication device according to claim 14, wherein the indication device further comprises a scale extending from the base and arranged on or behind the indication pin, where markings are provided on the scale facing the indication pin.

17. Indication device according to claim 15, wherein the indication device further comprises a scale extending from the base and arranged on or behind the indication pin, where markings are provided on the scale facing the indication pin.

18. Indication device according to claim 16, where the scale is provided to estimate the movement of the elongated indication pin, where the scale is adapted to measure an angular movement of the elongated pin and/or a longitudinal movement of the elongated pin and/or a rotational movement of the elongated pin where the scale is showing the movement of the elongated pin as an absolute measurement in either distance or in angular pivoting or in rotation or as an interval of movement.

19. Indication device according to claim 17, where the scale is provided to estimate the movement of the elongated indication pin, where the scale is adapted to measure an angular movement of the elongated pin and/or a longitudinal movement of the elongated pin and/or a rotational movement of the elongated pin where the scale is showing the movement of the elongated pin as an absolute measurement in either distance or in angular pivoting or in rotation or as an interval of movement.

20. Indication device according to claim 14, wherein the movement of the indication pin is indicated with colours on a scale showing a range of displacement in either distance or angular pivoting or rotation.

21. Indication device according to claim 14, where a scale is provided, said scale having adhesive means suitable to adhere the scale to the surface of the body.

22. Indication device according to claim 14, where the indication device comprises two or more connected elongated indication pins connected to separate bases, where the indication pins are connected by one or more bridge members, a first bridge member having a fixed or adjustable length between the two indication pins, and a second or more bridge member(s) having an adjustable but fixable length between the two indication pins.

23. Indication device according to claim 14, wherein the base is flexible and may be deformed to fit closely to the body, and where the indication pin is rigid.

24. Indication device according to claim 14, where the base is further provided with tear lines or very thin/weak sections, such that upon removal of the base, the base will tear along the tear lines or the thin/weak sections.

25. Indication device according to claim 14, where the base is attached to the desired body part by percutaneous or transcutaneous fixation directly into any skeleton bone.

26. Method of using an indication device for investigating an articulated body joint or body joint complex, wherein said indication device comprises an elongated indication pin connected to a base, said base suitable to be adhered to the skin of a body, where the base is adhered to a body part adjacent an articulated body joint, such that the indication pin overlays the articulated body joint or body joint complex.

27. Method according to claim 26, wherein a scale is applied to the body part between the indication pin and the body joint or body joint complex, where the scale is applied prior to movement of the articulated body joint or body joint complex.

28. Method according to claim 27, where the scale is a thin film having printed indications, said indications particular to the particular articulated body joint or body joint complex which is being investigated.

29. Method according to claim 27, where the scale is printed or indicated directly on the body.

30. Method according to claim 26, where the indication device is applied on a human body or to an animal body, in particular horses, dogs, cats, cattle, fish and aquatic mammals.
